(19) United States
(12) Patent Application Publication
(10) Pub. No.: US 2020/0249377 A1
(43) Pub. Date: Aug. 6, 2020

(54) DIFFRACTIVE OPTICAL DEVICE, ENDOSCOPIC PROBE, AND FABRICATION METHODS THEREFOR

(71) Applicant: Canon U.S.A., Inc., Melville, NY (US)

(72) Inventor: Maiko Niwa, Tokyo (JP)

(21) Appl. No.: 16/778,702

(22) Filed: Jan. 31, 2020

Related U.S. Application Data
(60) Provisional application No. 62/799,325, filed on Jan. 31, 2019.

(51) Int. Cl. G02B 5/18; A61B 1/06

(52) U.S. Cl. CPC G02B 5/1852; G02B 2005/1804; A61B 1/0646

(57) ABSTRACT

A diffractive optical device comprising: a base substrate; a resin layer having consecutively formed grating grooves and ridges, the resin layer being provided on a surface of the base substrate and being formed of a resin including a photo-curing resin or a thermosetting resin; and a side-surface resin layer. The side-surface resin layer is formed continuously with the resin layer and on a side surface of the base substrate so as to intersect end portions of the grating grooves and ridges, the side-surface resin layer being formed of a resin, which is a same material as that which forms the resin layer. An average thickness of the side-surface resin layer is greater than or equal to 0.1 μm and less than or equal to 35 μm. An endoscopic probe including the diffractive optical device, and a method of fabricating such device are disclosed.

### CROSS-REFERENCE TO RELATED APPLICATIONS

The present application claims priority to U.S. provisional application 62/799,325, filed Jan. 31, 2019, the disclosure of which is incorporated by reference herein in its entirety.

### BACKGROUND INFORMATION

### Field of Disclosure

The present disclosure generally relates to diffractive optics, and more specifically, it relates to diffractive optical devices manufactured by replica molding using a photo-curing resin or a thermosetting resin. A diffractive optical device may be used in optical apparatuses, such as video or still cameras, or ultra small endoscope cameras and endoscope optical probes.

### Description of Related Art

Composite optical elements that are obtained by replica molding of photo-curing or thermosetting resins are widely used in optical systems of, for example, small endoscope cameras, video and picture cameras, or other optical apparatuses. Specific examples of such composite optical elements include aspherical lenses, pickup lenses, and diffractive optical devices such as gratings and prisms. In a diffraction grating device, such as a diffraction grating, a plurality of grating elements (a grating pattern) having continuously and consecutively formed grating structures (e.g., grooves and ridges) arranged on a transparent substrate are used to cause diffraction of light. In this type of diffractive optical devices, the diffraction efficiency, which is the ratio of light which can be diffracted with respect to the total light incident on the diffractive optical device, is determined by the grating pattern. Generally, high diffraction efficiency is preferred. Accordingly, achieving high diffraction efficiency is a matter of a prime importance in designing and manufacturing a high-quality diffractive optical device.

Various methods of manufacturing such diffractive optical devices have been proposed. For example, Japanese Patent Application Laid-Open No. 2012-183753 (herein “Patent Literature 1”) discloses a method of manufacturing an optical element by a nanoimprint method. In the method of manufacturing a diffractive optical device by the nanoimprint method described in Patent Literature 1, the diffractive optical device has fine grating elements on a surface of a base substrate, which are formed of a cured resin layer integrated with the base substrate by successively performing the following steps. First, a step of filling a portion between a mold having fine grating elements and the base substrate with a resin material by dripping the material onto the mold or the base substrate is performed. Then, a step of curing the material, with which the portion between the mold and the base substrate has been filled, by applying curing energy is performed. Afterwards, a step of integrating the cured material and the base substrate with each other, and releasing the integrated diffractive optical device from the mold is performed.

However, in the diffractive optical device described in PTL 1, when the region of the base substrate where the resin layer is molded is very small, e.g., a few millimeters (mm) at most, and an aspect ratio of the consecutively formed grating elements is large, an interface between the base substrate and the resin layer may peel under high temperatures due to differences between the thermal expansion of the resin layer and the thermal expansion of the base substrate and due to the release of residual stress remaining in the resin layer when the material has been cured.

In addition, in a diffractive optical device, where the size of the base substrate to which the grating pattern is to be transferred is smaller than the size of the mold, surplus resin unevenly protrudes to an outer peripheral portion of the surface of the base substrate when the resin layer is pressed by the mold. When the resin is cured in the state in which the surplus resin protrudes, a part of the resin remains uncured, and, after the mold is separated, the uncured resin flows by capillary action into the fine grating pattern that has just been transferred. Then, sticking of grating elements adjacent to each other occurs due to the capillary action of the uncured resin. Thus, the pattern transfer and the diffraction efficiency of the grating are compromised.

The mechanism of occurrence of transfer failure is described below referring to FIG. 1A and FIG. 1B. FIG. 1A is a sectional view of a diffraction grating 120 having a repetitive pattern of linear ridges 121 (first grating elements) and grooves 122 (second grating elements) arranged periodically on a base substrate 123. The diffraction grating 120 is formed on a surface of the base substrate such that the linear ridges 121 and grooves 122 are arranged substantially equidistantly and substantially parallel to one another at a predetermined pitch P. The diffraction grating 120 can be made of curable or thermosetting resin using a mold having an inverse shape of the desired grating pattern. As shown in FIG. 1A, the diffraction grating 120 has a normal continuous rectangular shape formed substantially without defects. FIG. 1B is a sectional view of the diffraction grating 120 having a transfer failure 124. The inventors herein have found that a transfer failure, as that shown in FIG. 1B, tends to occur when two adjacent grating ridges 121 get stuck to each other due to a residue of resin 125 that has flowed by capillary action into a grating groove 122 of the diffraction grating 120. This capillary action often tends to occur at the edge portion of the diffractive optical device on a side surface of the base substrate in a direction in which the grating elements are continuously formed and adjacent to an end portion of the base substrate.

In view of the above state of the art, it is difficult to reproduce the intended grating patterns accurately. In addition, because high-precision molds are difficult and expensive to produce, an effective method for transferring miniature diffractive structures without distortion remains a major challenge. Moreover, even when proper transfer is achieved, high temperature environments ultimately cause peeling of resin-based pattern layer.

### SUMMARY OF INVENTION

Accordingly, an object of the present disclosure is to provide a diffractive optical device which achieves high-diffraction efficiency and makes it possible, even under high temperatures, to prevent the peeling at an interface between the base substrate and the resin layer. To that end, a diffractive optical device of the present disclosure is provided with consecutively formed grating elements provided on a base substrate and formed of a photo-curing resin or a thermosetting resin, and is provided with a resin layer formed on a side surface of the base substrate in a direction in which the grating elements are continuously formed and adjacent to an end portion of the base substrate, and formed of the same component as the resin that forms the grating elements.

A method of manufacturing a diffractive optical device includes: forming a side-surface layer of curable resin or thermosetting resin on a side-surface of a surface of a base substrate; forming a resin layer of the same curable resin or thermosetting resin on the surface of the base substrate; pressing the resin layer with a mold having an inverse shape of a grating pattern to be transferred to the resin layer; curing the pressed resin layer to form a diffraction grating; and demolding the diffraction grating when the curable resin is in a cured state in which the base substrate and the resin layer are integrated with each other. The side-surface layer is formed continuously with the resin layer and on the side-surface of the base substrate in a direction in which the grating elements are continuously formed, the side-surface resin layer being formed of the same material as that which forms the resin layer; and a thickness of the side-surface layer is greater than or equal to 0.1 μm and less than or equal to 35 μm.

According to the present disclosure, it is possible to provide a diffractive optical device that has high diffraction efficiency and is capable of sufficiently suppressing the occurrence of peeling at an interface between the base substrate and a resin layer under high temperatures. According to a diffractive optical device disclosed in the present application, side-surface resin layers are provided in a direction transverse to end portions of grating elements formed on a resin layer to improve the adhesive property between the resin layer and a base substrate.

### BRIEF DESCRIPTION OF DRAWINGS

FIG. 1A and FIG. 1B are explanatory diagrams showing an occurrence of transfer failure due to capillary action;

FIG. 2A shows a top view of a diffractive optical device, FIG. 2B shows a sectional view taken along line A, and FIG. 2C shows a sectional view taken along line B of the diffractive optical device.

FIGS. 3A, 3B, 3C, and 3D show sectional views of the diffractive optical device having side-surface resin layers according to the present disclosure.

FIG. 4 shows a partial sectional view of the diffractive optical device and dimensional parameters thereof, according to the present disclosure.

FIG. 5A shows an exemplary 3-dimensinal representation of a base substrate having a square cross section. FIG. 5B is a top view of the square surface of the base substrate.

FIG. 6A shows an exemplary 3-dimensinal representation of a base substrate having a cylindrical or semi-cylindrical cross section. FIG. 6B is a top view of the circular or semicircular surface of the base substrate.

FIGS. 7A, 7B, 7C, 7D, and 7E illustrate various examples of dimensions and location of the side-surface resin layer with respect to the surface of the base substrate.

FIGS. 8A, 8B, 8C, and 8D show exemplary steps of a method of manufacturing the diffractive optical device according to an embodiment of the present disclosure.

FIGS. 9A, 9B, 9C, and 9D show exemplary steps of a method of manufacturing a diffractive optical device on an optical probe, according to an example 10 of the present disclosure.

FIGS. 10A, 10B, and 10C illustrate a schematic view of an endoscopic optical probe including a diffractive optical device according to an embodiment of the present disclosure.

FIG. 11 illustrates a schematic diagram of an exemplary imaging system.

### DETAILED DESCRIPTION OF EMBODIMENTS

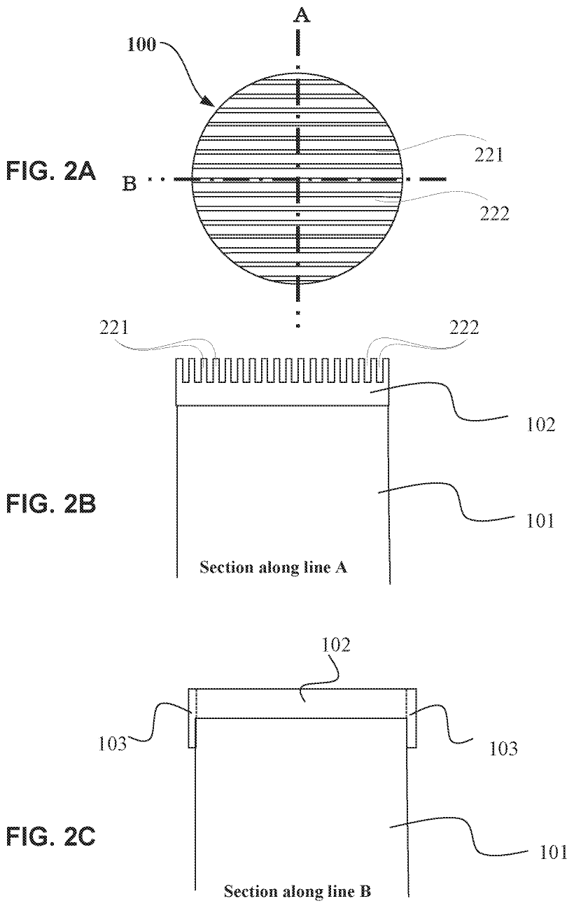

FIG. 2A shows a top view of a diffractive optical device 100, FIG. 2B shows a sectional view taken along line A, and FIG. 2C shows a sectional view taken along line B of the diffractive optical device 100, according to an embodiment of the present disclosure. The diffractive optical device 101 of the present disclosure is a diffractive optical device that includes a base substrate 101 and a resin-based grating layer 102. The resin-based grating layer 102 (hereinafter “resin layer 112”) is formed on a surface of the base substrate 101. As shown in FIG. 2A, the diffractive optical device 100 includes a plurality of ridges 221 (first grating elements) and a plurality of grooves 222 (second grating elements) arranged substantially parallel to each other at a predetermined pitch. The ridges 221 and grooves 222 extend in a longitudinal direction parallel to a line B, and are arranged parallel to each other in a width direction thereof along line A. The width direction along line A is perpendicular to the longitudinal direction along line B.

FIG. 2B is a sectional view when the top view shown in FIG. 2A is cut in the direction of a straight line A. FIG. 2B shows a structure in which the resin-based grating layer 102 is formed having consecutively rectangular linear structures (rectangular ridges 221 and rectangular grooves 222) formed on the surface of the base substrate 101. FIG. 2C is a sectional view when the top view shown in FIG. 2B is cut in the direction of a straight line B in which one linear structure (one ridge) formed on the surface of the base substrate 101 is cut along its longitudinal direction. FIG. 2C shows side-surface resin layers 103 formed on side surfaces of the base substrate 101. The side-surface resin layer 103 is formed of the same resin material as the resin layer forming the grating structure. Preferably, the side-surface resin layer 103 is formed in a direction which intersects the end portions of the grating grooves 222 and ridges 221 continuously arranged next to each other; that is, the side-surface resin layer 103 is formed adjacent to end portions of the resin layer such that the side-surface resin layer 103 is formed continuously with the resin layer 102. In other words, the side-surface resin layer 103 is formed at both end portions of the grating elements and on the periphery (adjacent to end portions) of the resin layer. Here, that the “side-surface resin layer is formed continuously with the resin layer” indicates that the side-surface resin layer 103 and the resin layer 102 are, at least partially, in physical contact with each other so as to form a continuous structure.

As materials containing the photo-curing resin or the thermosetting resin, materials having material characteristics, such as the proper refractive index, transmissivity, viscosity, and curing shrinkage ratio, so as to acquire the desired optical characteristics and good moldability may be selected. More specifically, examples of the thermosetting resin include epoxy resin, and examples of the photo-curing resin include acrylic resin, epoxy resin, and fluororesin.

Although the drawings of FIGS. 2A, 2B, and 2C show grating elements in the shape of rectangular ridges 221 and grooves 222, the grating elements are not particularly limited to certain shapes as long as the shapes of grating elements satisfy the desired optical characteristics that are calculated from the optical system and material characteristics. The width 106 of recessed portions or grooves 222 and the width 105 of protruding portions or ridges 221 may be the same or one of the widths may be larger than the other. The corners of the grating elements (grooves and ridges) are not limited to only right-angled corners, and may be chamfered corners, curved corners, or saw-tooth-shaped corners. As shown in FIGS. 2A and 2B, the resin layer 102 having the grating grooves 222 and ridges 221 is formed on a cross-sectional surface of the base substrate 101. The surface of the substrate where the resin-based grating is formed is referred to as the optically effective surface. The side-surface resin layer 103, as shown in FIG. 2C, refers to the layer of resin formed on a region of the substrate 101 other than the optically effective surface. An optically effective surface of the grating elements is not necessarily limited to a planar surface, and may be, for example, spherical or aspherical regardless of the shape of the base substrate.

As the base substrate 101, those formed of materials or having shapes used in ordinary optical apparatuses, such as flat glasses, glass lenses, or lenses made of thermoplastic resin, may be used. In particular, as the base substrate 101, those having surface shapes that are planar, spherical, or aspherical may be suitably used. In addition, for example, a thin film that increases adhesion with the resin layer or a film that provides reflection prevention may be formed on the surface of the base substrate.

The direction in which the grating elements are continuously formed is a direction in which the grating elements that exist at the optically effective portion are continuously formed, and, as shown in FIG. 2A, is a direction that is parallel to the straight line B in a structure that is continuous along the straight line. This continuous direction may be along a curve or a concentric circular shape, in which case the direction in which the grating elements are continuously formed also indicates a curve or a concentric circular shape.

The diffractive optical device too of the present disclosure includes the side-surface resin layers 103 that are disposed on the side surfaces of the base substrate 101. The side-surface resin layers 103 are each located at a portion where a line indicating the direction in which the grating elements are continuously formed and the corresponding end portion of the base substrate intersect each other. The side-surface resin layers 103, which are formed on the side surfaces of the base substrate 101, are formed of resin, which is the same material as the material that contains photo-curing resin or thermosetting resin of which the grating elements are formed.

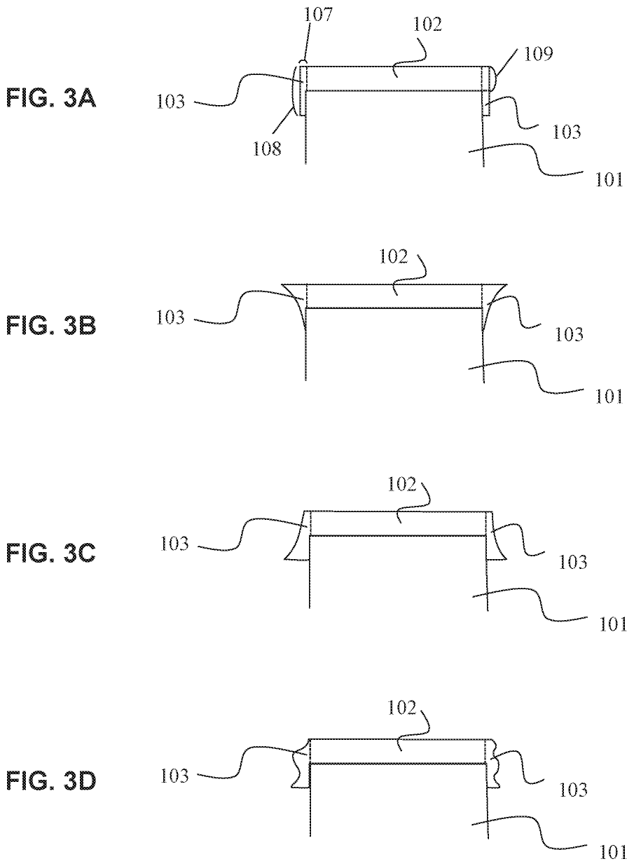

FIGS. 3A, 3B, 3C and 3D show sectional views taken along the longitudinal direction of one grating element of the diffractive optical device of the present disclosure. As shown in FIG. 3A, the side-surface resin layers 103 may have flat shapes. Alternatively, the following shapes may be suitably formed. For example, as shown in FIG. 3B, the side-surface resin layers 103 may have shapes whose thickness decreases along the end portions; as shown in FIG. 3C, the side-surface resin layers 103 may have shapes whose thickness increases along the end portions; or as shown in FIG. 3D, the side-surface resin layers 103 may have shapes that undulate considerably along the side of base substrate 101.

Since stress that is generated due to differences between the thermal expansion of the base substrate 101 and the thermal expansion of the resin layer 102 is largest at the end portions and shrinkage stress when photo-curing resin or thermosetting resin is cured becomes largest at the end portions, peeling at an interface between the base substrate and the resin layer of the diffractive optical device under high temperatures start from the end (or edge) portions of the resin layer. Further, when the aspect ratio of the grating elements is large and the height of the protruding portions of the grating elements becomes 1 μm or greater, the peeling at the interface occurring at the end portions tends to progress in the longitudinal direction in which the grating elements are formed.

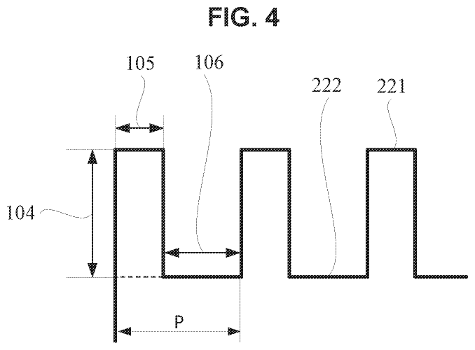

FIG. 4 shows an enlarged view of the grating elements of the diffractive optical device 100 of the present disclosure. The term “aspect ratio” here refers to a ratio obtained by dividing a height 104 of a protruding portion or ridge 221 by the smaller of a width 105 of the protruding portion or a width 106 of a recessed portion or grove 221. The aspect ratio is desirably greater than or equal to 3 and less than or equal to 10. When the aspect ratio becomes less than or equal to 3, the progress of peeling at the interface of resin layer and substrate can increase quickly and may become noticeable. In contrast, when the aspect ratio becomes greater than or equal to 10, since shrinkage stress of resin when the grating elements are being formed becomes large, the grating elements (ridges 221) may crumble onto each other or break when releasing the resin layer from the mold. As a result, it may become difficult to realize the desired optical performance.

It is also desirable that the height 104 of each protruding portion be greater than or equal to 1 μm and less than or equal to 3 μm. When the height 104 of each protruding portion is less than 1 μm, peeling or breaking (cracking) at an interface of resin layer and substrate also tends to progress quickly. On the other hand, when the height 104 of each protruding portion is greater than 3 μm, an increase in the shrinkage stress of the resin when forming the grating elements causes the grating elements to randomly crumble onto each other and/or and the grating elements can break when releasing the resin layer from a mold. As a result, it may be difficult to realize the desired optical performance.

Peeling at an interface between the base substrate and the resin layer that has occurred at an end portion progresses in a longitudinal direction in which the grating elements are formed, and the peeling can reach an optically effective portion. As a result, the optical performance of diffractive optical device deteriorates. In particular, when the size of the optical device is very small, e.g., in the range of a few mm or less, the distance from the edge (end portion) of the resin layer to the optically effective portion becomes very small. Therefore, the deterioration in the optical performance caused by the progress in the peeling at the interface of resin layer and substrate that has occurred at the end portion of the grating elements becomes noticeable.

When the side-surface resin layers 103 are formed on the side end portions of the base substrate that are positioned in a direction in which the grating elements are continuously formed, the area of the interface between the base substrate and the resin layer increases, so that it is possible to increase adhesion properties between the base substrate and the resin layer. When the side-surface resin layers 103 are formed, the location of concentration of stress on the end portions that is generated under high temperatures changes to end portions of the side-surface resin layers 103, so that it is possible to prevent interface peeling at an grating shaped section at an initial stage. In addition, since the side-surface resin layers 103 that are formed on the respective side surface portions of the base substrate do not have grating elements, the interface peeling in a direction in which the grating elements are continuously formed does not progress, as a result of which even if interface peeling at the end portions of the side-surface resin layers 103 may occur, the peeling does not progress. Therefore, deterioration in optical performance caused by the peeling at the interface between the base substrate and the resin layer can be prevented.

As mentioned above, the peeling at the interface, which occurs when the optical element is placed under high temperatures, starts when peeling occurs at the end portions of the grating elements, and is caused by the progress of the peeling along the grating structure. In order to prevent the progress of the peeling along the grating structure, the inventor(s) herein have found out that it is effective to form resin layers on the side surface portions of the base substrate that contact the end portions of the grating grooves and ridges. This prevents the occurrence of peeling at the interface under high temperatures, so that it is possible to apply grating elements having a high aspect ratio and including relatively tall protruding portions to small optical components.

The average of a thickness 107 of each side-surface resin layer is greater than or equal to 0.1 μm and less than or equal to 35 μm. When the thickness is less than or equal to 0.1 μm, adhesion between the base substrate and the side-surface resin layers 103 is not sufficient, as a result of which peeling of the grating elements is no longer sufficiently prevented. In contrast, when the thickness is greater than or equal to 35 μm, the overall size of the optical element is increased, as a result of which the optical element can no longer be used as, in particular, an element where, for example, small cameras for endoscopes are required to be smaller. More desirably, the average of the thickness 107 of each side-surface resin layer 103 is greater than or equal to 0.1 μm and less than or equal to 20 μM.

In order to prevent peeling of the optically effective portion of the resin layer, it is desirable that each side-surface resin layer 103 be formed on a region that is greater than or equal to 80% of the area surrounding of the surface of the base substrate 101 on which the diffraction grating is formed. More specifically, the progress of peeling to the optically effective portion generally starts at the edge of the surface of the base substrate where the grooves and ridges of the grating end. To prevent the start of peeling at the edge of optically effective portion, the side-surface resin layer 103 is formed as explained above. However, to improve the adhesive property between the resin layer and base substrate, while still maintaining high diffraction efficiency of the diffractive optical device, it is necessary to use an appropriate amount of resin in forming the side-surface resin layer. The amount of resin will depend on the geometrical parameters and physical shape of the base substrate 101, as well as the area of the surface of the base substrate on which the grating is to be formed. Optical grade glass or quartz rods can be fabricated in many shapes including round, square, rectangular and oval shapes. When each side-surface resin layer 103 is formed on a region that is less than or equal to 80% the area of the side-surface, since peeling at an interface occurs between the base substrate and the resin layer at the edge of the optically effective portion, the optical element may not satisfy the required optical performance of the diffractive optical device.

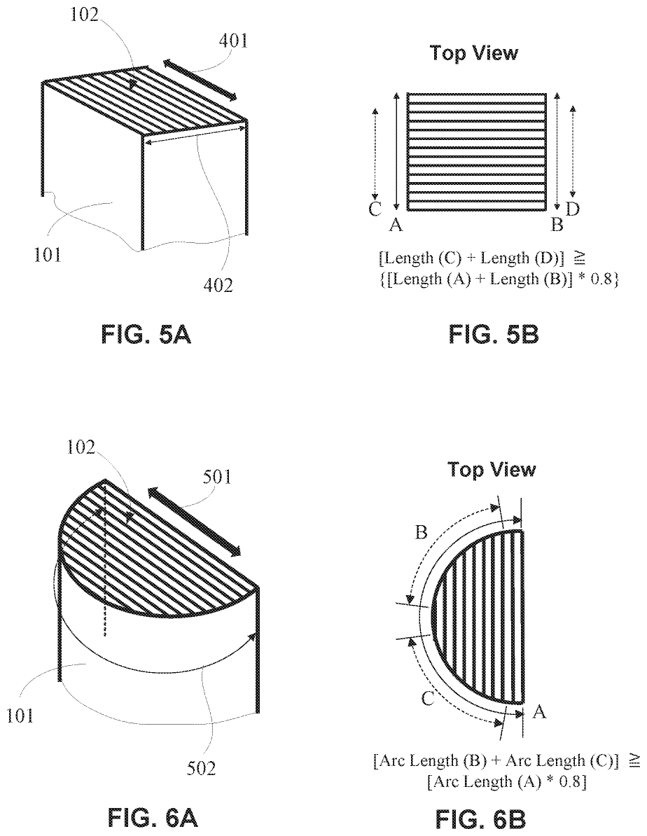

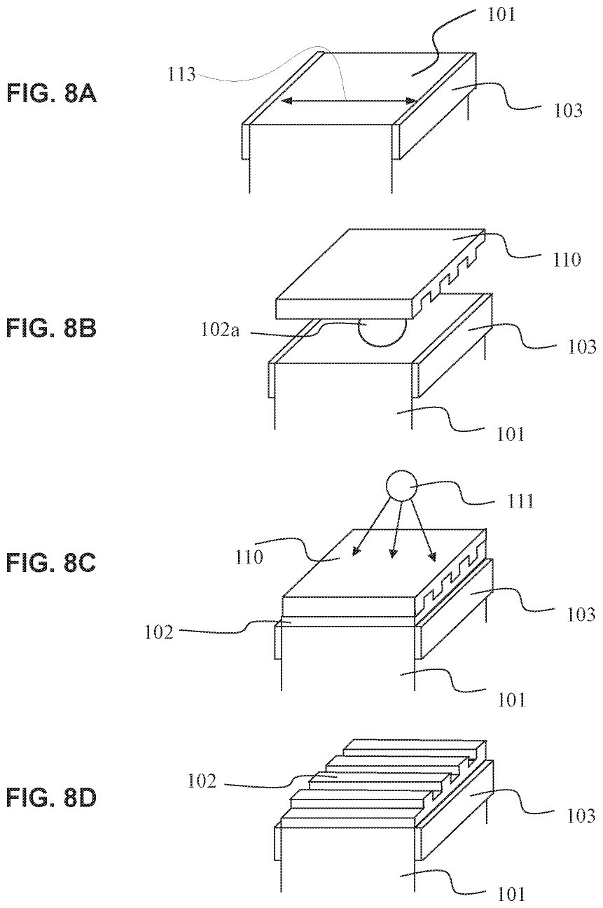

FIG. 5A shows an exemplary 3-dimensinal representation of a base substrate 101 for a case where a grating is formed on a surface of a square rod (embodiment of FIGS. 8A-8D). FIG. 5B is a top view of the square surface of the base substrate 101 on which a resin layer 102 having grating grooves and ridges has been formed. In the case of a square rod, grating grooves and ridges are arranged in a longitudinal direction 401 which is parallel to one side of the square surface. The direction 401 is a direction in which the grating grooves and ridges are linearly continuous. In this case, the side-surface resin layer 103 is to be formed in a direction 402 perpendicular to the direction 401 along the linear side-surface of base substrate 101 such that the side-surface resin layer 103 intersects transversally the end portions of the grooves and ridges. And to ensure prevention of peeling, the length of the grating edge where the grating grooves meet the side-surface is larger than the length of the side-surface resin layer. More specifically, as shown in FIG. 5B, as an example, (C) and (D) are the areas where the side surface of the base substrate 101 has the side-surface resin layer 103, and (A) and (B) correspond to the length of the grating edge where the grating grooves meet the side-surface. Therefore, here, it is necessary to satisfy the condition [Length (C)+Length (D)]≥{[Length (A)+Length (B)]*0.8}.

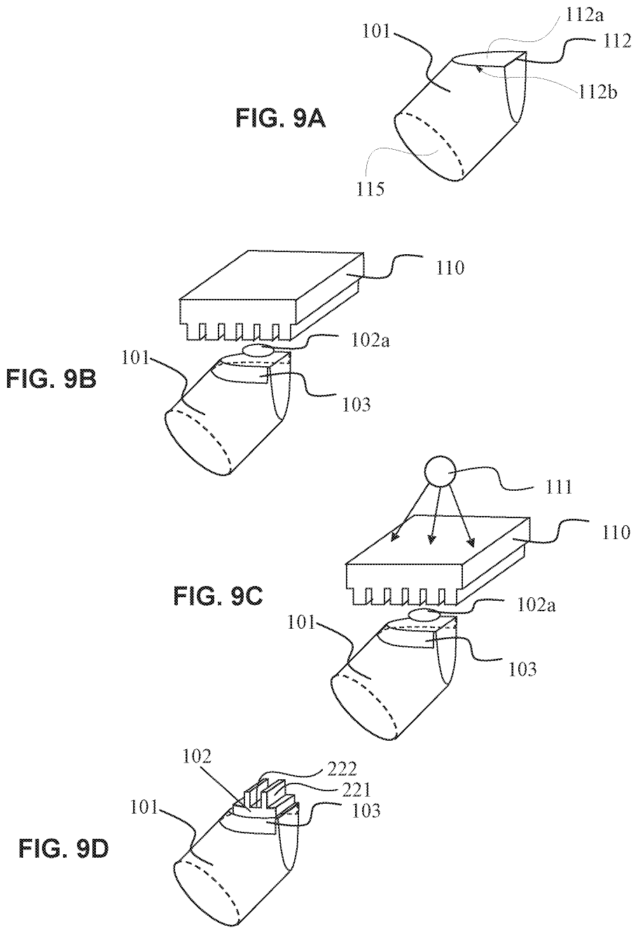

FIG. 6A shows an exemplary 3-dimensinal representation of a base substrate 101 for a case where a grating is formed on a surface of a cylindrical or semi-cylindrical or oval rod (e.g., embodiment of FIGS. 9A-9D). FIG. 6B is a top view of the circular or semicircular surface of the base substrate 101 on which a resin layer 102 having grating grooves and ridges has been formed. In the case of a cylindrical rod, grating grooves and ridges are arranged in a longitudinal direction 501 parallel to the diameter of the cylindrical rod. The direction 501 is a direction in which the grating grooves and ridges are linearly continuous. In this case, the side-surface resin layer 103 is to be formed in a curved direction 502 along the curved side-surface of base substrate 101 such that the side-surface resin layer 103 intersects the end portions of the grooves and ridges at an angle. And to ensure prevention of peeling, the arc length of the grating edge where the grating grooves meet the side-surface is larger than the length of the side-surface resin layer. More specifically, as shown in FIG. 6B, as an example, (B) and (C) are the areas where the side surface of the substrate 101 has the side-surface resin layer, and A is the arc length of the surface of the base substrate where the grating is formed. Therefore, here, it is necessary to satisfy the condition: [Arc Length (B)+Arc Length (C)]≥[Length (A)*0.8]. From FIG. 5B and FIG. 6B, it is shown that the resin layers on the corresponding side surface portions of the base substrate are formed on, of the end portions of the base substrate in a direction along the grating elements ends, a region that is 80% or greater thereof.

It is desirable that the average of the thickness 107 of each side-surface resin layer 103 shown in FIG. 3A be less than a length 108 of each side-surface resin layer 103. When the average of the thickness 107 is greater than the length 108, stress caused by curing shrinkage when forming the side-surface resin layers 103 is increased. As a result, peeling of the side-surface resin layers 103 from the base substrate 101 tends to occur. In addition, when the length 108 is too small (e.g. close to the average thickness 107), peeling of the side-surface resin layer may occur up to the end portions of the grating elements; as a result of which it may be difficult to sufficiently prevent deterioration in optical performance.

It also is desirable that the average of the thickness 107 of each side-surface resin layer 103 be less than the average of a thickness or height 109 of the resin layer 102 having the grating elements. When the thickness 107 of each side-surface resin layer 103, which is formed on the corresponding side surface portion of the base substrate 101, is less than the thickness or height 109 of the resin layer 102 having the grating elements, concentration of stress caused by differences between the thermal expansion of the base substrate and the thermal expansion of each side-surface resin layer 103 under high temperatures is reduced. As a result, peeling at an interface occurring at the end portions of the side-surface resin layers 103 can be effectively prevented.

The method of manufacturing the side-surface resin layers 103 is not limited to any particular method. As long as resin layers 103 can be formed on side surfaces of the base substrate 101 on the periphery of the optically effective portion, side-surface resin layers can be formed by various methods and in different shapes (e.g., as shown in FIGS. 3A-3D).

For example, the side-surface resin layers 103 may be formed by applying a proper amount of resin to the side surfaces of the base substrate such that the resin is formed into a desired shape, and by curing the resin. As means of applying the resin to the side surfaces of the base substrate, for example, a dispenser may be used. In order to precisely form the shape of each side-surface resin layer, it is possible to provide a mask on each side surface of the base substrate. After curing the resin, and by a cutting operation or an acid treatment operation, it is possible to control the desired shape of each resin layer. In order to increase adhesion between the side resin layers 103 and the base substrate 101, it is possible to apply a silane coupling solution on the side surfaces of the base substrate.

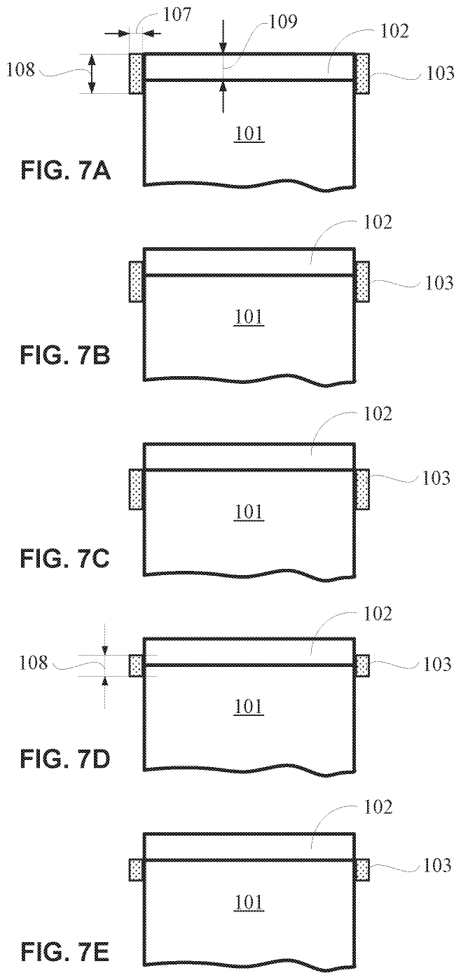

FIGS. 7A-7E illustrate various examples of dimensions and location of the side-surface resin layer 103 with respect to the surface of the base substrate 101. As illustrated in FIG. 7A, the side-surface resin layers 103 may be formed with a length 108 extending from the top edge of the resin layer 102 (grating) and passing beyond the interface of resin layer 102 and edge of substrate 101. As illustrated in FIG. 7B, the side-surface resin layers 103 may be formed with a length 108 extending from below the edge of the resin layer 102 (grating) and passing beyond the interface of resin layer 102 and edge of base substrate 101. As illustrated in FIG. 7C, the side-surface resin layers 103 may be formed with a length 108 extending from approximately the bottom edge of the resin layer 102 (i.e., the side-surface resin layers 103 may start at the interface of resin layer 102 and edge of substrate 101) along the side surface of the base substrate 101 in the longitudinal direction of the base substrate. In all cases of FIGS. 7A, 7B and 7C, the length 108 of the side-surface resin layer 103 is larger than the height or thickness 109 of the resin layer 102; and the thickness 107 of the side-surface resin layer 103 is smaller than both the length 108 and height 109.

In certain embodiments, the length 108 of the side-surface resin layer 103 can be comparable to the height or thickness 109 of the resin layer 102. For example, as illustrated in FIGS. 7D and 7E, the side-surface resin layer 103 can have a length 108 which is substantially equal to (or even smaller than) the height or thickness 109 of the resin layer 102. In FIG. 7D, the side-surface resin layer 103 extends from below the edge of the resin layer 102 (grating) and passes beyond the interface of resin layer 102 and edge of base substrate 101. In FIG. 7E, the side-surface resin layer extends from approximately the bottom edge of the resin layer 102 (i.e., the side-surface resin layers 103 may start at the interface of resin layer 102 and edge of substrate 101) along the side surface of the base substrate 101 in the longitudinal direction of the base substrate. In all of the cases illustrated in FIGS. 7A-7E, the side-surface resin layer 103 is formed continuously with the resin layer and on a side surface of the base substrate 101 in a direction which intersects end portions of the grating grooves and ridges formed in the resin layer 102.

### Manufacturing Examples

FIGS. 8A-8D and FIGS. 9A-9D illustrate exemplary embodiments of a method of manufacturing a diffractive optical device. The method of manufacturing a diffractive optical device according to the present disclosure includes several steps, as graphically summarized in FIGS. 8A-8D. FIG. 8A shows a first step of forming, on each side of the base substrate 101, a side-surface resin layer 103 on at least a side surface portion thereof. Specifically, the side-surface resin layer 103 is formed in portion of the base substrate where a line 113 that indicates a longitudinal direction in which grating elements are to be formed on the base substrate intersects an end portion (edge) of the base substrate. In other words, in the illustration of FIG. 8A, the side-surface resin layer 103 is formed on a side surface of the base substrate and in a direction transversal to the longitudinal direction in which grating elements are to be formed. FIG. 8B shows a second step of filling a portion (a volumetric space) between the mold 110 and the base substrate 101 with a material 102a containing photo-curing resin or thermosetting resin. The filling step includes a first sub-step of dripping a desired amount of material 102a; and a second sub-step of pressing the material 102a by moving one or both of the mold 110 and the substrate 101 towards each other. The mold 110 has a shape which is an inverted shape of the desired grating elements to be formed.

FIG. 8C shows a third step of curing the material toga by applying thereto curing energy (heat or light) with an irradiation device 111 to form a resin layer 102. FIG. 8D shows a fourth step of releasing the cured material (releasing the resin layer 102) integrated with the base substrate 101 from the mold 110, and thereby forming the diffractive optical device. Here, the side-surface resin layers 103 formed on the corresponding side surface portions of the base substrate 101 are made of a material that is the same as the material containing photo-curing resin or thermosetting resin forming the grating elements in the resin layer 102.

### Example 1

A diffractive optical device of the present example and a method of manufacturing the diffractive optical device are described by referring to FIGS. 8A-8D. FIGS. 8A, 8B, 8C, and 8D show exemplary steps of a method of manufacturing the diffractive optical device according to an embodiment of the present disclosure.

For the base substrate 101, a lens made of glass and whose both surfaces were substantially planar surfaces 1 mm square and 2 mm long was used. For the resin layer 102 having the grating elements and the side-surface resin layers 103 on the corresponding side surface portions of the base substrate, a coating liquid whose main component was photo-curing fluororesin was used.

The mold 110 has a shape that is an inverted shape of a diffraction grating that realizes the desired optical performance. As the material of the mold, publicly known materials, such as a metal, a resin, silicone, or quartz may be used. For example, the mold no may be one manufactured by performing etching on quartz. Alternatively, for example, a mold formed using a resin material from a master mold may be used. A publicly known thin film may be formed on a surface of the mold. Examples of the thin film include a releasing film, such as a nitride film and a DLC (Diamond-Like Carbon) film, and films coated with releasing agents, such as silicone-based releasing agents, fluorine-based releasing agents, or non-silicone-based releasing agents. Here, in example 1, a mold having a fluorine-based releasing film provided on the mold manufactured by performing etching on silicone was used.

The grating elements of the diffractive optical device to be manufactured is such that each protruding portion or ridge has a height of 1.5 μm and a width of 0.3 μm, a grating period is 2 μm, and an aspect ratio is 5.

First, silane coupling for increasing adhesion with the materials was performed on the entire front surface and side surfaces of the base substrate. Next, each side-surface resin layer 103 was formed on its corresponding side surface of the base substrate positioned at an end portion in a height direction of the grating structure formed on the base substrate. Using a dispenser, 3 pl (picoliters) of the aforementioned coating liquid whose main component was photo-curing fluororesin was applied to the side surface portions of the base substrate and was irradiated with ultraviolet light in a vacuum to form the side-surface resin layers 103 thereon. The thickness of each side-surface resin layer 103 formed on its corresponding side surface portion of the base substrate (in a transverse direction in a plane of FIG. 8A) was 2 μm to 3 μm, and the average thickness thereof was 2.3 μm. The length of each side-surface resin layer 103 (in a vertical direction in a plane of FIG. 8A) was 0.5 mm. Although it is possible to control the thickness of each side-surface resin layer 103 on the basis of only the coating amount by the dispenser, the thickness may be adjusted by providing a fixing jig at the base substrate with a moving mechanism as appropriate.

After the side-surface resin layer 103 was formed, as shown in FIG. 7B, 12 pl of a coating liquid whose main component was photo-curing fluororesin was dripped on a location near the center of the surface of the base substrate 101 where the grating elements were to be formed. Next, the mold 110 was disposed above the base substrate 101 on which the coating liquid was dripped, and the mold 110 and the base substrate 101 were gradually brought close to each other, to fill a portion (volumetric space) between the base substrate 101 and the mold 110 with the coating liquid. Here, significant pressing was not performed on the mold.

Next, as shown in FIG. 8C, using an ultraviolet-ray irradiation device 111 (UL-750 (product name) manufactured by HOYA CANDEO OPTRONICS), the coating liquid was irradiated with ultraviolet (UV) light for 200 seconds, and was subjected to photo-curing. Thereafter, a force was applied to an outer peripheral portion of the mold 110 in a releasing direction from the base substrate to release the base substrate 101 and the cured resin layer 102 having the grating elements from the mold (FIG. 8D). The average thickness of the resin layer 102 having the grating elements molded on the base substrate 101 was 10 μm, and the diffraction efficiency of the obtained diffractive optical device satisfied the desired performance.

The manufactured diffractive optical device was left standing for two hours at 70° C., and its durability was confirmed. No changes in the diffraction efficiency, corresponding to an optical characteristic, were observed before and after keeping the diffractive optical device under high temperatures, and peeling between the base substrate and the resin layer having the grating elements did not occur.

In this way, according to the example 1, a diffractive optical device in which the occurrence of peeling between the base substrate and the resin layer under high temperatures was suppressed and in which the optical performance was satisfied was obtained. Therefore, it is possible to install the diffractive optical device in an optical apparatus, such as a camera or a video, and expect that peeling or breakage will not occur under high temperature performance.

### Comparative Example 1

In a diffractive optical device of a Comparative Example 1, side-surface resin layers 103 were not formed on corresponding side surface portions of a base substrate. Although the optical performance of the obtained diffractive optical device did not differ from that of Example 1, peeling at an interface occurred between the base substrate and a resin layer having grating elements after keeping the diffractive optical device under high temperatures, as a result of which the optical performance considerably deteriorated and a diffraction phenomenon could not be observed.

### Example 2

A diffractive optical device of Example 2 was manufactured by the same method as Example 1 except that the amount of coating liquid dripped on side surfaces was 2 pl. In the diffractive optical device of Example 2, each side-surface resin layer 103 formed on its corresponding side surface portion of a base substrate was formed on, of the corresponding side surface of the base substrate where the side-surface resin layer 103 contacted its corresponding end portion of a grating shape, a region that was 80% thereof. The end portions of the grating elements refer to the end portions in a transverse direction in a plane of FIG. 8D. The side-surface resin layers 103 of Example 2 are such that the near side and the far side in a depth direction in a plane of FIG. 8D are each formed in a region that is 10% shorter compared to those of the side-surface resin layers 103 of Example 1. Although the optical performance of the obtained diffractive optical device did not differ from that of Example 1, a deterioration of 0.5% in the optical characteristics after keeping the diffractive optical device under high temperatures was observed. This is due to peeling at an interface between the base and a resin layer having the grating elements. However, a deterioration of 0.5% in the optical performance is not a serious problem in terms of the characteristics of the diffractive optical device, and is within a deterioration range that allows the diffractive optical device to be installed in an optical apparatus, such as a camera or a video.

### Example A

A diffractive optical device of Example A was manufactured by the same method as Example 1 except that the amount of coating liquid dripped on side surfaces was 1.8 pl. In the diffractive optical device of Example A, each side-surface resin layer formed on its corresponding side surface portion of a base substrate was formed on, of the corresponding side surface of the base substrate where the resin layer contacted its corresponding end portion of a grating shape, a region that was 75% thereof. The end portions of the grating elements refer to the end portions in a transverse direction in a plane of FIG. 8D. The side-surface resin layers 103 of Example A are such that the near side and the far side in the depth direction in the plane of FIG. 8D are each formed in a region that is 12.5% shorter compared to those of the side-surface resin layers 103 of Example 1. Although the optical performance of the obtained diffractive optical device did not differ from that of Example 1, a deterioration of 5% in the optical characteristics after keeping the diffractive optical device under high temperatures was observed. This is due to peeling at an interface between the base substrate and a resin layer having the grating elements. In the images of optical apparatuses, such as cameras and videos, having the diffractive optical device installed therein, slight effects of scattering and flares were observed.

Table 1 below shows the region where each resin layer was formed on the corresponding side surface portion of the base substrate that exists at the corresponding end portion of the base substrate in a direction along the grating structure, and changes in the optical characteristics after placing the diffractive optical device under high temperatures. Table 1 shows that when the resin layers on the corresponding side surface portions of the base substrate are formed on, of the end portions of the base substrate in a direction along the grating elements, a region that is 80% or greater thereof, a diffractive optical device having the required high optical characteristics as an optical apparatus, such as a camera or a video, even after the diffractive optical device was left standing under high temperatures is manufactured.

TABLE 1

| | Region where side- | Optical performance after keeping |
|---|---|---|
| | surface resin layers | diffractive optical device |
| | are formed | under high temperatures |
| Example 1 | 100% | No changes |
| Example 2 | 80% | Slight deterioration (−0.5%) |
| Example A | 75% | Slight deterioration (−5%) |
| Comparative | None | Large deterioration (diffraction |
| Example 1 | | phenomena does not occur) |

### Example 3

A diffractive optical device of Example 3 was manufactured by the same method as Example 1 except that the amount of coating liquid dripped on side surfaces was 2 pl. In the diffractive optical device of Example 3, the length of each side-surface resin layer 103 (in a vertical direction in the plane of FIG. 8A) on its corresponding side surface portion of a base substrate was 5 μm. The optical performance of the obtained diffractive optical device did not differ from that of Example 1, and no changes occurred in the optical characteristics after keeping the diffractive optical device under high temperatures. However, peeling at an interface between the base substrate and a resin layer having grating elements occurred though the peeling occurred outside an optically effective portion.

### Example B

A diffractive optical device of Example B was manufactured by the same method as Example 1 except that the amount of coating liquid dripped on side surfaces was 0.05 pl. In the diffractive optical device of Example B, the length of each side-surface resin layer 103 (in a vertical direction in the plane of FIG. 8A) on its corresponding side surface portion of a base substrate was 1 μm. Although the optical performance of the obtained diffractive optical device did not differ from that of Example 1, a deterioration of 30% in the optical characteristics after keeping the diffractive optical device under high temperatures was observed. This is due to peeling at an interface between the base substrate and a resin layer having grating elements. In the images of optical apparatuses, such as cameras and videos, having the diffractive optical device installed therein, slight effects of scattering and flares were observed.

### Example 4

A diffractive optical device of Example 4 was manufactured by the same method as Example 1 except that the amount of coating liquid dripped on side surfaces was 1.5 pl. In the diffractive optical device of Example 4, the average thickness of each side-surface resin layer 103 (in a transverse direction in the plane of FIG. 8A) on its corresponding side surface portion of a base substrate was 15 μm, and the length thereof (in a vertical direction in the plane of FIG. 8A) was 50 μm. The optical performance of the obtained diffractive optical device did not differ from that of Example 1, and no changes in the optical characteristics occurred after keeping the diffractive optical device under high temperatures. Peeling at an interface between the base substrate and a resin layer having grating elements did not occur.

### Example C

A diffractive optical device of Example C was manufactured by the same method as Example 1 except that the amount of coating liquid dripped on side surfaces was 0.3 pl. In the diffractive optical device of Example C, the average thickness of each side-surface resin layer 103 (in a transverse direction in the plane of FIG. 8A) on its corresponding side surface portion of a base substrate was 15 μm, and the length thereof (in a vertical direction in the plane of FIG. 8B) was 10 μm. Although the optical performance of the obtained diffractive optical device did not differ from that of Example 1, a deterioration of 25% in the optical characteristics after keeping the diffractive optical device under high temperatures was observed. This is due to peeling at an interface between the base substrate and a resin layer having grating elements. In the images of optical apparatuses, such as cameras and videos, having the diffractive optical device installed therein, slight effects of scattering and flares were observed.

Table 2 below shows the thicknesses of the resin layers formed on the corresponding side surface portions of the base substrate that exist at the end portions of the base substrate in a direction along the grating structure, the lengths thereof, and changes in the optical characteristics after placing the diffractive optical device under high temperatures. Table 2 shows that when the thicknesses of the resin layers on the corresponding side surface portions of the base substrate are less than the lengths thereof, a diffractive optical device having the required high optical characteristics as an optical apparatus, such as a camera or a video, even after being left standing under high temperatures is manufactured.

TABLE 2

| | Thicknesses of | Lengths of | |
|---|---|---|---|
| | resin layers | resin layers | Optical performance |
| | on side surface | on side surface | after keeping diffractive |
| | portions of | portions of | optical device under |
| | base substrate | base substrate | high temperatures |
| Example 1 | 2.3 μm | 500 μm | No changes |
| Example 3 | 2.3 μm | 5 μm | No changes |
| | | | (peeling occurred |
| | | | outside optically |
| | | | effective portion) |
| Example B | 2.3 μm | 1 μm | Deteriorated (−30%) |
| Example 4 | 15 μm | 50 μm | No changes |
| Example C | 15 μm | 10 μm | Deteriorated (−25%) |

### Example 5

A diffractive optical device of Example 5 was manufactured by the same method as Example 1 except that the amount of coating liquid dripped on side surfaces was 3 pl. In the diffractive optical device of Example 5, the average thickness of a resin layer having grating elements was 30 μm. The optical performance of the obtained diffractive optical device did not differ from that of Example 1, and no changes occurred in the optical characteristics after keeping the diffractive optical device under high temperatures.

### Example 6

A diffractive optical device of Example 6 was manufactured by the same method as Example 5 except that the amount of coating liquid dripped on side surfaces was 20 pl. In the diffractive optical device of Example 6, the thickness of each side-surface resin layer 103 on its corresponding side surface portion of a base substrate was 20 μm. The optical performance of the obtained diffractive optical device did not differ from that of Example 1, and no changes occurred in the optical characteristics after keeping the diffractive optical device under high temperatures. Peeling at an interface between the base substrate and a resin layer having grating elements did not occur. However, since the size of the diffractive optical device as an optical element was large at a range of 2 mm to 2.04 mm, there was concern about an increase in the entire size when using the optical element in a small camera for an endoscope.

### Example D

A diffractive optical device of Example D was manufactured by the same method as Example 5 except that the amount of coating liquid dripped on side surfaces was 27 pl. In the diffractive optical device of Example D, the thickness of each side-surface resin layer 103 on its corresponding side surface portion of a base substrate was 27 μm. The optical performance of the obtained diffractive optical device did not differ from that of Example 1, and no changes occurred in the optical characteristics after keeping the diffractive optical device under high temperatures. Peeling at an interface between the base substrate and a resin layer having grating elements did not occur. However, since the size of the diffractive optical device as an optical element was large at a range of 2 mm to 2.05 mm, there was concern about an increase in the entire size when using the optical element in a small camera for an endoscope.

### Example 7

A diffractive optical device of Example 7 was manufactured by the same method as Example 5 except that the amount of coating liquid dripped on side surfaces was 0.2 pl. In the diffractive optical device of Example 7, the thickness of each side-surface resin layer 103 formed on its corresponding side surface portion of a base substrate was 0.17 μm. The optical performance of the obtained diffractive optical device did not differ from that of Example 1, and no changes occurred in the optical characteristics after keeping the diffractive optical device under high temperatures. Peeling at an interface between the base substrate and a resin layer having grating elements did not occur.

### Comparative Example 2

A diffractive optical device of Comparative Example 2 was manufactured by the same method as Example 5 except that the amount of coating liquid dripped on side surfaces was 0.05 pl. In the diffractive optical device of Comparative Example 2, the thickness of each side-surface resin layer 103 on its corresponding side surface portion of a base substrate was 0.06 μm. Although the optical performance of the obtained diffractive optical device did not differ from that of Example 1, a deterioration of 70% in the optical characteristics after keeping the diffractive optical device under high temperatures was observed. This is due to peeling at an interface between the base substrate and a resin layer having grating elements. In the images of optical apparatuses, such as cameras and videos, having the diffractive optical device installed therein, the effects of scattering and flares were observed.

Table 3 below shows the thicknesses of the resin layers formed on the corresponding side surface portions of the base substrate existing at the end portions of the base substrate in a direction along the grating structure, changes in the optical characteristics after placing the diffractive optical device under high temperatures, and applicability to small endoscope cameras. Table 3 shows that when the thicknesses of the resin layers on the corresponding side surface portions of the base substrate are in a range of 0.1 μm to 20 μm, a diffractive optical device that has the required optical characteristics as an optical apparatus, such as a camera or a video, even after being left standing under high temperatures and that does not cause the size of the entire optical apparatus to be increased is manufactured.

TABLE 3

| | Thicknesses of | Optical | |
|---|---|---|---|
| | side-surface | characteristics | |
| | resin layers | after keeping | Applicability |
| | on side surface | diffractive | to small |
| | portions of | optical device under | endoscope |
| | base substrate | high temperatures | cameras |
| Example 5 | 2.3 μm | No changes | Yes |
| Example 6 | 20 μm | No changes | Yes |
| Example D | 27 μm | No changes | Yes |
| Example 7 | 0.17 μm | No changes | Yes |
| Comparative | 0.06 μm | Deteriorated (−70%) | Yes |
| Example 2 | | | |

### Example 8

A diffractive optical device of Example 8 was manufactured by the same method as Example 1 except that the amount of coating liquid dripped on side surfaces was 10 pl. In the diffractive optical device of Example 8, the average thickness of each side-surface resin layer 103 on its corresponding side surface portion of a base substrate was 9.2 μm. The optical performance of the obtained diffractive optical device did not differ from that of Example 1, and no changes occurred in the optical characteristics after keeping the diffractive optical device under high temperatures. Peeling at an interface between the base substrate and a resin layer having grating elements did not occur.

### Example E

A diffractive optical device of Example E was manufactured by the same method as Example 8 except that the amount of coating liquid dripped on side surfaces was 20 pl. In the diffractive optical device of Example E, the average thickness of each side-surface resin layer 103 on its corresponding side surface portion of a base substrate was 18 μm. The optical performance of the obtained diffractive optical device did not differ from that of Example 1, and no changes occurred in the optical characteristics after keeping the diffractive optical device under high temperatures. Peeling at an interface between the base substrate and a resin layer having grating elements did not occur.

### Example 9

A diffractive optical device of Example 9 was manufactured by the same method as Example 1 except that the amount of coating liquid dripped on side surfaces was 20 pl. In the diffractive optical device of Example 9, the average thickness of each side-surface resin layer 103 on its corresponding side surface portion of a base substrate was 18 μm. The optical performance of the obtained diffractive optical device did not differ from that of Example 1, and no changes occurred in the optical characteristics after keeping the diffractive optical device under high temperatures. Peeling at an interface between the base substrate and a resin layer having grating elements did not occur.

### Example F

A diffractive optical device of Example F was manufactured by the same method as Example 1 except that the amount of coating liquid dripped on side surfaces was 35 pl. In the diffractive optical device of Example F, the average thickness of each side-surface resin layer 103 on its corresponding side surface portion of a base substrate was 34 μm. The optical performance of the obtained diffractive optical device did not differ from that of Example 5, and no changes occurred in the optical characteristics after keeping the diffractive optical device under high temperatures. Peeling at an interface between the base substrate and a resin layer having grating elements did not occur.

Table 4 below shows the thickness of the resin layer having grating elements, the thicknesses of the resin layers formed on the corresponding side surface portions of the base substrate that exist at the end portions of the base substrate in a direction along the grating structure, and changes after placing the diffractive optical device under high temperatures. Table 4 shows that, when the average thickness of each resin layer on its corresponding side surface portion of the base substrate is less than the average thickness of the resin layer having grating elements, peeling does not occur even at the resin layers formed on the corresponding side surface portions of the base substrate, which is desirable.

TABLE 4

| | | Thicknesses | |
|---|---|---|---|
| | | of side-surface | Occurrence |
| | Thickness | resin layers on | of peeling |
| | of resin | side surface | between base |
| | layer having | portions of base | substrate and |
| | grating elements | substrate | resin layer |
| Example 1 | 10 μm | 2.3 μm | None |
| Example 8 | 10 μm | 9.2 μm | None |
| Example E | 10 μm | 18 μm | None |
| Example 5 | 30 μm | 2.3 μm | None |
| Example 9 | 30 μm | 18 μm | None |
| Example F | 30 μm | 34 μm | None |

### Example 10

A diffractive optical device according to Example 10 is described referring to FIGS. 9A-9D. In this example, for a base substrate 101, a cylindrical rod formed from Tempax (product name made of borosilicate glass; manufactured by Schott), and having two surfaces formed by cutting one end of the cylindrical rod is used. As shown in FIG. 9A, the cylindrical rod base substrate 101 has a proximal end 115 and a distal end 112, a diameter Φ=0.5-mm, a length l=1.5 mm. A surface where a diffraction grating is to be formed is one of the two surfaces formed by cutting the distal end of the cylindrical rod in the shape of a wedge-shaped prism. In one implementation, the base substrate 101 can be a modified Powell lens. The Powell lens design is disclosed in U.S. Pat. No. 4,826,299 and, according to the present example, the Powell lens can be adopted to fabricate the diffractive optical device one surface of a wedge formed at one end of the cylindrical rod, and then shaping the one surface of the wedge as the surface where the diffractive optical device is formed.

For a resin layer 102 having grating elements and a side-surface resin layer 103 on a corresponding side surface portion of the base substrate 101, materials containing photo-curing acrylic resin as a main component and having high transmissivity with respect to visible light can be used. For the mold no, a mold in which a fluorine-based releasing film was formed on quartz having a shape that was an inverse shape of the diffraction grating realizing the desired optical performance was used.

The grating elements of the diffractive optical device to be manufactured are such that each ridge or protruding portion has a height of 1.8 μm and a width of 1.7 μm. The groove or recessed portion is formed to satisfy a grating period P=2 μm, and an aspect ratio of 6.

In this example, first, silane coupling for increasing adhesion with the materials was performed on the entire front surface and side surface of the base substrate 101. The diffraction grating is to be formed such that the grating elements (ridges and grooves) are parallel to an edge line of the distal end 112. The edge line being the line between the surfaces of the wedge-shaped prism formed by the cutting of the cylindrical rod. And, an end portion of the base substrate where the side-surface resin layer 103 is to be formed corresponds to a curved portion 112b (side surface) of the surface 112a (main surface) where the grating structure is to be formed.

With the above considerations, first, in order to form the side-surface resin layer 103 on the curved portion 112b, using a dispenser, 0.5 pl (pico liter) of a coating liquid was applied to the curved portion.

Thereafter, as shown in FIG. 9B, 1 pl of the resin material 102a was dripped on a location near the center of the surface 112a of the base substrate 101 where the grating elements were to be formed. Next, the mold 110 was disposed above the base substrate 101 on which the material was dripped, and the mold 110 and the base substrate 101 were gradually brought close to each other to fill a volumetric space between the base substrate and the mold with the resin material.

The entire base substrate 101, the coating liquid that filled the surface 112a and curved portion 112b, and the mold 110 were placed under a nitrogen atmosphere. Then, as shown in FIG. 9C, using an ultraviolet-ray irradiation device in (EXECURE-H-1 (product name) manufactured by HOYA CANDEO OPTRONICS), the base substrate 101, mold 110, and resin material 102a were irradiated with ultraviolet light for 100 seconds, and were subjected to photo-curing, as illustrated in FIG. 9C. Here, the material previously applied to the end portion in the direction of the grating structure can also be cured to form the side-surface resin layer 103 on the corresponding side surface portion of the base substrate.

Thereafter, a force was applied to an outer peripheral portion of the mold 110 in a releasing direction (away from the base substrate 101) to release from the mold 110 the cured resin layer 102 having the grating elements integrated with base substrate 101. The released cured resin layer 102 having the grating elements integrated with base substrate 101 is shown in FIG. 9D. The average thickness of the resin layer 102 having the grating elements molded on the base substrate 101 was 8 μm, and the diffraction efficiency of the obtained diffractive optical device satisfied the desired performance. The side-surface resin layer 103 on its corresponding side surface portion of the base substrate had a thickness of 1 to 4 μm (with an average thickness of about 3 μm), and a length of 0.1 mm (or 100 μm). The side-surface resin layer 103 was formed on, of the corresponding side surface portion of the base substrate positioned at its corresponding end portion in a structural direction of the grating structure, a region that was 90% thereof.

The manufactured diffractive optical device was left standing for two hours at 70° C., and its durability and performance were confirmed experimentally. No change in the diffraction efficiency, corresponding to an optical characteristic, was observed before and after keeping the diffractive optical device under high temperatures, and peeling between the base substrate and the resin layer having the grating elements did not occur.

In this manner, according to the present example, a diffractive optical device in which the occurrence of peeling between the base substrate and the resin layer under high temperatures was suppressed, and in which the optical performance was satisfied was provided. Therefore, it is possible to install the diffractive optical device in an optical apparatus, such as a camera or an endoscope.

### Example 11

Using a mold having a shape differing from the shape of the mold used in Example 10, a diffractive optical device of Example 11 was manufactured with the form of a diffraction grating in which the height of each protruding portion of an grating shape was 2.8 μm and the width thereof was 0.3 μm, a grating period was 2 μm, and an aspect ratio was 9.3. Changes in the optical characteristics of the obtained diffractive optical device before and after keeping the diffractive optical device under high temperatures were not observed, and peeling at an interface between a base substrate and a resin layer having grating elements did not occur.

### Example 12

Using a mold having a shape differing from the shape of the mold used in Example 10, a diffractive optical device of Example 12 was manufactured with the form of a diffraction grating in which the height of each protruding portion of an grating shape was 2.8 μm and the width thereof was 1.7 μm, a grating period was 2 μm, and an aspect ratio was 9.3. Changes in the optical characteristics before and after keeping the diffractive optical device under high temperatures were not observed, and peeling at an interface between a base substrate and a resin layer having the grating elements did not occur.

### Example 13

Using a mold having a shape differing from the shape of the mold used in Example 10, a diffractive optical device of Example 13 was manufactured with the form of a diffraction grating in which the height of each protruding portion of an grating shape was 1.8 μm and the width thereof was 0.6 μm, a grating period was 2 μm, and an aspect ratio was 3. Changes in the optical characteristics of the obtained diffractive optical device before and after keeping the diffractive optical device under high temperatures were not observed, and peeling at an interface between a base substrate and a resin layer having the grating elements did not occur.

### Endoscopic Optical Probe

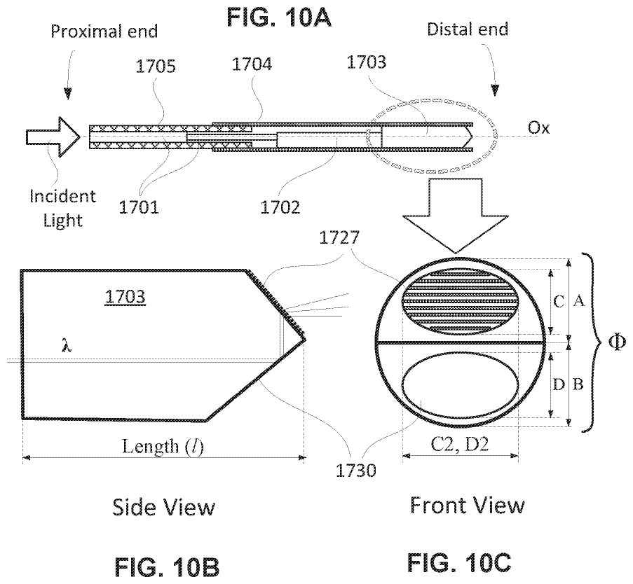

FIGS. 10A-10C show an exemplary endoscopic optical probe. FIG. 10A is a schematic view of an endoscopic optical probe applicable to a spectrally encoded endoscope (SEE) to which a diffractive optical device according to any of the above embodiments can be applied. FIG. 10B is a side view of a cylindrical rod (a spacer) including the diffractive optical device, and FIG. 10C is a front view of the distal end of the optical probe as seen in the direction from the distal end to the proximal end of the probe.

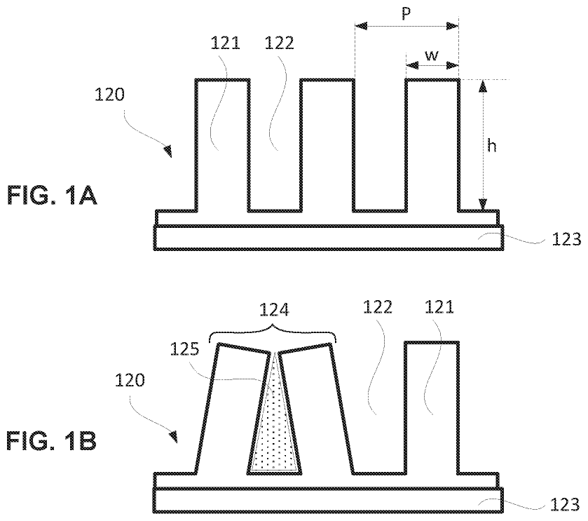

An endoscopic optical probe of this exemplary SEE endoscope includes, in order from the proximal end to the distal end, an illumination optical system 1701, a GRIN lens 1702, and a spacer 1703 arranged along a probe axis Ox. The illumination optical system 1701 is connected, at the proximal end thereof, to a rotary junction and to a light source (shown in FIG. 1i). Light emitted from the light source is transmitted to the rotary junction. The illumination optical system 1701 receives light from the rotary junction and transmits the light to an end portion thereof. The end portion of the illumination optical system 701 is connected to the GRIN lens 1702. The GRIN lens 1702 functions as a focusing element which focuses the light, received from the illumination optical system 1701, to a region on or near an object. The end portion of the illumination optical system 1701, the GRIN lens 1702, and the spacer 1703 are disposed inside a transparent protection tube 1704 (a sheath). The endoscopic optical probe is rotated around the probe axis Ox by the rotary junction via a drive shaft 1705. As the probe rotates, illumination light transmitted through the spacer 1703, is reflected by a mirror surface 1730, is diffracted by a grating pattern 1727 into a spectrally encoded line, and irradiates a target object located at a predetermined working distance (Wd). Since the optical probe rotates, the spectrally encoded line also rotates on the object and scans a two-dimensional region of the object. Thus, a two-dimensional region (a circular region) of the object can be imaged by the endoscopic optical probe.

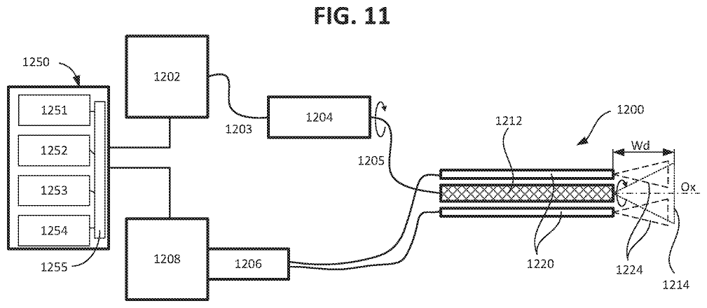

In one embodiment, the endoscopic optical probe including the diffractive optical device is used only for emitting illumination light, and a light-collecting optical system for collecting light back-scattered by the object is independently provided. In this case, optical fibers (detecting fibers) are used to collect the light scattered by the object and to transmit the collected light back to a detector or spectrometer. For example, the optical fibers are arranged in a ring so as to surround the transparent protection tube 1704, as further explained with reference to FIG. 11. In another embodiment, the endoscopic optical probe may be used as an illumination optical system and as a light-collecting optical system.

In the endoscopic optical probe of FIG. 10A, the spacer 1703 is a cylindrical rod made of substantially transparent material, and it includes the grating pattern 1727 molded according to any of the processes, embodiments, and examples, or according to a combination of the examples described above. According to one example, the length (l) of the spacer 1703 is about 2 mm; and the diameter is Φ=0.5 mm. The distal end of the spacer is shaped into a triangular or wedge-shaped prism having two surfaces joined at the distal end 112, as described above. A first surface is a reflecting surface having the mirror surface 1730 and a second surface is a diffraction surface having the grating pattern 1727. In one example, as shown in FIG. 10C, the second surface has a dimension A=250 μm and the first surface has a dimension B=250 μm. That is, each side surface of the triangular prism occupies half of the diameter of the cylindrical rod. The diffraction surface (second surface) has an ellipsoidal shape having a minor axis C=225 μm and a major axis C2=320 μm. The reflecting surface (first surface) also has an ellipsoidal shape having a minor axis D=225 μm and a major axis D2=320 μm. On the diffraction surface, the grating pattern 1727 includes a groove-and-ridge pattern where a width of each groove is about 0.462 μm, a depth of each groove is about 1.8 μm, and a pitch between two consecutive grooves is about 1.54 μm.

### Imaging System

FIG. 11 illustrates an exemplary imaging system equipped with an endoscopic SEE probe, according to an embodiment of the present disclosure. The system of FIG. 11 includes, for example, an endoscopic probe 1200, a broadband light source 1202, a fiber optic rotary juntion FORJ 1204, a spectrometer 1206, a detector 1208, and a computer 1250. A fiber 1203 connects the broadband light source 1202 to the fiber rotary junction 1204. Light or other electromagnetic radiation goes from theh ligh source to the FORJ 1204, then light goes from the FORJ 1204 through an optical fiber (illumination fiber 1205); the illumination fiber 1205 extends along an inner sheath 1212 from the proximal end to the distal end of the probe 1200. A rotation or oscillation of the illumination fiber 1205 along with the rotation of the inner sheath 1212 and a cone of illumiation light 1214 is depicted with the circular arrows. The cone of illumination light 1214, which is formed by the rotation of the spectrally encoded line, is incident on a sample (not shown) at a working distance Wd. A plurality of detection fibers 1220 are arranged in a ring pattern to surround the inner sheat 1212 at least at the distal end thereof. Light collected by a cone of acceptance 1224 of each dection fiber 1220 is delivered to the spectrometer 1206 and/or the detector 1208. Specifically, light is sent through the plurality of detection fibers 1220 (e.g., multimode fibers), which, at the proximal end of the probe 1200, are lined up in a linear array at the entrance slit to the spectrometer 1206. The light (dispersed light) is then imaged on the detector 1208. The computer 1250 controls the overal operations of the light source 1202, FORJ 1204, and detector 1208 to obtain an image of a non-illustrated sample. In some embodiments, the detector 1208 can be a line scan sensor, such as a line scan camera.

Certain aspects of the various embodiment(s) of the present disclosure can be realized by one or more computers that reads out and executes computer executable instructions (e.g., one or more programs) recorded on a transitory or non-transitory storage medium to perform the functions of one or more of the above-described embodiment(s) and/or that includes one or more circuits for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

For example, in FIG. 1i, the computer 1250 may include central processing unit (CPU) 1251, a storage memory (RAM) 1252, a user input/output (I/O) interface 1253, and a system interface 1254, which are interconnected by a system bus 1255. The computer 1250 can issue a command that can be transmitted to the imaging system via the system interface 1254. A touch panel screen can be included as part of the user interface unit 1253, in addition a key board, mouse, joy-stick, ball controller, and foot pedal can also be included as part of the user interface. The user can cause a command to be initiated to observe inside a lumen of a human body through the exemplary front-viewing SEE probe using the user interface unit/imaging processor. For example, when the user inputs a command via the user interface 11253, the command is transmitted to the CPU 1251 for execution thereby causing the CPU to issue a command via the system interface 1254 to one or more of the light source 1202, the detector 1208, spectrometer 1206, or the FORJ 1204. The CPU 1251 is comprised of one or more processors (microprocessors) configured to read and perform computer-executable instructions stored in the storage memory 1252.

### Definitions

In referring to the description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error. The term “substantially”, as used herein means that, within fabrication parameters and/or measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated.

The term “average” refers to the arithmetic mean. Therefore, the “average thickness” of each side-surface resin layer refers to the arithmetic mean of the layer thickness. Similarly, the “average height” of the grating ridges of a diffraction grating formed on the resin layer refers to the arithmetic mean of the height of the grating ridges.

The foregoing merely illustrates the principles of the disclosure. Various modifications and alterations to the described exemplary embodiments will be apparent to those skilled in the art in view of the teachings herein. Indeed, the arrangements, systems and methods according to the exemplary embodiments of the present disclosure can be used with any SEE system or other imaging systems.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

1: A diffractive optical device comprising:
a base substrate;
a resin layer having consecutively formed grating grooves and ridges substantially parallel to one another at a predetermined pitch, the resin layer being provided on a surface of the base substrate and being formed of a resin material including a photo-curing resin or a thermosetting resin; and
a side-surface resin layer,
wherein the side-surface resin layer is formed continuously with the resin layer and on a side surface of the base substrate so as to intersect end portions of the grating grooves and ridges, the side-surface resin layer being formed of a same material as the resin material which forms the resin layer, and
wherein an average thickness of the side-surface resin layer is smaller than a thickness of the resin layer.

2: The diffractive optical device according to claim 1,
wherein an average thickness of the side-surface resin layer is greater than or equal to 0.1 μm and less than or equal to 35 μm.

3: The diffractive optical device according to claim 1,
wherein a thickness of the resin layer formed on the surface of the substrate is greater than or equal to 10 μm and less than or equal to 30 μm.

4: The diffractive optical device according to claim 1,
wherein the surface of the base substrate has an edge that intersects the end portions of the grating grooves and ridges, and
wherein the side-surface resin layer is formed along the edge that intersects the end portions on a region of the side surface where the length of the edge with side-surface resin formation is greater than or equal to 80% of the total length of the edge where the grating grooves and ridges intersect the side surface of the base substrate.

5: The diffractive optical device according to claim 1,
wherein the surface of the base substrate has an edge that intersects the end portions of the grating grooves and ridges, and
wherein the side-surface resin layer is formed along the edge on a region of the side surface where an average thickness of the side-surface resin layer measured from the edge is less than a length by which the side-surface resin layer contacts the side surface of the base substrate.

6: The diffractive optical device according to claim 5, wherein the average thickness of the side-surface resin layer is greater than or equal to 0.1 μm and less than or equal to 20 μm.

7: The diffractive optical device according to claim 1,
wherein an average thickness or height of the resin layer is greater than an average thickness of the side-surface resin layer.

8: The diffractive optical device according to claim 1,
wherein an average height of the grating ridges of a diffraction grating formed on the resin layer is greater than or equal to 1 μm and less than or equal to 3 μm, and an aspect ratio of the diffraction grating is greater than or equal to 3 and less than or equal to 10.

9: The diffractive optical device according to claim 1,
wherein the substrate is made of glass and has a square cross section, and
wherein the grating grooves and ridges are formed parallel to one side of the square cross section, and the side-surface resin layer is formed on a side surface of the base substrate perpendicular to the one side.

10: The diffractive optical device according to claim 1,
wherein the substrate is made of glass and has a circular or semi-circular cross section, and
wherein the grating grooves and ridges are formed parallel to the diameter of the circular or semi-circular cross section, and the side-surface resin layer is formed along the curved surface of the circular or semi-circular cross section.

11: The diffractive optical device according to claim 10,
wherein the side-surface resin layer is formed, on a side of the base substrate along the curved surface of the circular or semi-circular cross section so as to intersect the end portions of the grating grooves and ridges, on a region of the side surface that is greater than or equal to 80% of the curved surface.

12: An endoscopic probe comprising:
a light guiding component, a light focusing component, and a diffractive component enclosed within a sheath along an axis of the probe,
wherein the diffractive component comprises:
a base substrate;
a resin layer having consecutively formed grating grooves and ridges substantially parallel to one another at a predetermined pitch, the resin layer being provided on a surface of the base substrate and being formed of a resin material including a photo-curing resin or a thermosetting resin; and
a side-surface resin layer,
wherein the side-surface resin layer is formed continuously with the resin layer and on a side surface of the base substrate so as to intersect end portions of the grating grooves and ridges, the side-surface resin layer being formed of a same material as the resin material which forms the resin layer, and
wherein an average thickness of the side-surface resin layer is smaller than a thickness of the resin layer.

13: A method of manufacturing a diffractive optical device, comprising:
providing a base substrate having a main surface and a side surface;
forming, on the main surface of the substrate, a resin layer having consecutively arranged grating grooves and ridges substantially parallel to one another at a predetermined pitch, the resin layer being made of a resin material including a photo-curable resin or a thermosetting resin; and
forming, on the side surface of the base substrate, a side-surface layer made of a same material as the resin material which forms the resin layer,
wherein forming the resin layer on the main surface of the base substrate includes,
pressing the resin material with a mold having an inverse shape of a grating pattern to be transferred to the resin layer;
curing the pressed resin material to form a diffraction grating; and
demolding the diffraction grating when the curable resin is in a cured state in which the base substrate and the resin layer are integrated with each other, and
wherein forming the side-surface layer includes,
applying the same material as the resin material which forms the resin layer on the side surface of the base substrate continuously with the resin layer and in a direction which intersects end portions of the grating grooves and ridges of the diffraction grating, and
curing the resin material applied to the side surface,
wherein an average thickness of the side-surface resin layer is smaller than a thickness of the resin layer.
